(19) United States
(12) Patent Application Publication
(10) Pub. No.: US 2014/0004521 A1
(43) Pub. Date: Jan. 2, 2014

(54) MicroRNA-Based Methods for Prognosis of Hepatocellular Carcinoma

(71) Applicants: Chau-Ting Yeh, Taoyuan County (TW); Ya-Hui Huang, Taoyuan County (TW); Hua-Chien Chen, Taoyuan County (TW); Kwang-Huei Lin, Taoyuan County (TW)

(72) Inventors: Chau-Ting Yeh, Taoyuan County (TW); Ya-Hui Huang, Taoyuan County (TW); Hua-Chien Chen, Taoyuan County (TW); Kwang-Huei Lin, Taoyuan County (TW)

(21) Appl. No.: 13/539,786

(22) Filed: Jul. 2, 2012

(51) Int. Cl. C12Q 1/68; G06F 19/10

(52) U.S. Cl. 435/6.12; 702/19

(57) ABSTRACT

Disclosed herein is a method for determining the prognosis of a subject diagnosed with hepatocellular carcinoma based on the expression level of at least one microRNA in the non-cancerous liver tissue of the subject. According to various embodiments of the present disclosure, expression levels of miR-486-3p, miR-876-5p, and miR-381 are positively associated with a favorable prognosis, while expression levels of miR-30c, miR-432, and miR-15b are negatively associated with a favorable prognosis.

### BACKGROUND OF THE INVENTION

1. Field of the Invention

The present disclosure generally to the field of molecular biology. More particularly, the disclosure invention relates to the use of microRNAs to determine the prognosis of a subject diagnosed with hepatocellular carcinoma.

2. Description of Related Art

Hepatocellular carcinoma (HCC) is the fifth most common solid malignant tumors worldwide and the third leading cause of cancer related death. HCC arises most frequently in patients with inflammatory livers resulting either from hepatitis virus (such as hepatitis B virus or hepatitis C virus) infection or from metabolic disorders or toxic insults (e.g., the ingestion of alcohol or aflatoxin B1). Decisions on the therapeutic modalities are made largely according to the status of tumor growth at the time of diagnosis as well as the expected outcomes of the diseases.

MicroRNAs (miRNAs) are single-stranded non-coding RNAs of about 17 to 25 nucleotides in length. They are processed from longer (approximately 70 nucleotides in length) hairpin-like precursors termed pre-microRNAs. MicroRNAs assemble in complexes termed miRNAs and recognize their targets by antisense complementarity. If the microRNAs match 100% of their target, i.e., the complementarity is complete, the target mRNA is cleaved. If the match is incomplete, i.e., the complementarity is partial, then the translation of the target mRNA is blocked. MicroRNAs play important roles in many types of biological processes, such as organ development, cell differentiation and proliferation, cell apoptosis, and carcinogenesis. Accordingly, there is emerging research about the role of microRNAs in a variety of cancer diseases.

To date, identification of miRNAs differentially expressed between tumors and their non-tumor counterparts (e.g., the non-tumor tissue of the same patient or the non-tumor tissue of a healthy individual) is an approach adopted by most researchers. Such experiments have been conducted in HCC by a few groups with inconsistent results. Moreover, the differentially expressed miRNAs identified by this strategy should be considered as candidate diagnostic markers rather than prognostic predictors.

In view of the foregoing, a need remains to develop a prognostic tool that provides a sufficient resolution in assisting patient stratification for prognosis and/or therapy.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is based on the discovery that the expression levels of certain miRNAs, in a non-cancerous liver tissue of a subject, are positively or negatively associated with the clinical outcome of the subject.

In one aspect, the present disclosure is directed to a method for determining the prognosis of a subject diagnosed with hepatocellular carcinoma.

According to one embodiment of the present disclosure, the method comprises the steps as follows. In step (a), a non-cancerous sample is obtained from the non-cancerous liver tissue of the subject. Then, in step (b), the expression level (Ctₘᵢᵣ) of a microRNA in the non-cancerous sample is detected. The microRNA is at least one microRNA selected from a first group or a second group, in which the first group consists of miR-486-3p, mir-876-5p, and mir-381; whereas the second group consists of mir-30c, mir-432, and mir-15b. In step (c), the Ctₘᵢᵣ of the microRNA or a parameter derived therefrom is compared with a cutoff value associated with the microRNA for classifying the microRNA into (1) a high-expression type when the Ctₘᵢᵣ of the microRNA or a parameter derived therefrom is greater than the cutoff value associated with the microRNA, or (2) a low-expression type when the Ctₘᵢᵣ of the microRNA or a parameter derived therefrom is smaller than the cutoff value associated with the microRNA. The presence of a high-expression type microRNA from the first group indicates a favorable prognosis for the subject; while the presence of a low-expression type microRNA from the first group indicates an unfavorable prognosis for the subject. The presence of a low-expression type microRNA from the second group indicates a favorable prognosis for the subject; while the presence of a high-expression type microRNA from the second group indicates an unfavorable prognosis for the subject.

According to certain optional embodiments, the non-cancerous liver tissue is obtained from a paraneoplastic liver tissue of the subject.

In various optional embodiments, the method further comprises detecting the expression level (Ct_{ctrl}) of an internal control microRNA in the non-cancerous sample. For example, the internal control microRNA is mir-30d according to one embodiment of the present disclosure.

Still optionally, the Ctₘᵢᵣ of the respective microRNA may be normalized with the expression level (Ct_{ctrl}) of the internal control microRNA to obtain a normalized expression level (ΔCtₘᵢᵣ) of the microRNA. The normalized expression level (ΔCtₘᵢᵣ) of the microRNA may be used as the parameter derived from the respective microRNA, and is calculated according to equation (1):

normalized expression level=Ct_{ctrl}−Ctₘᵢᵣ equation (1).

In other optional embodiments, the parameter derived from the respective microRNA is an amplified expression level (amplified ΔCtₘᵢᵣ) of the microRNA calculated according to equation (1) above and equation (2):

amplified expression level=2^{ΔCtmir}×10⁶ equation (2).

For optional embodiments in which the internal control microRNA is mir-30d, and the amplified expression level (amplified ΔCtₘᵢᵣ) is used in the step (c) for comparison, the cutoff value for each of the microRNAs recited in the first and second groups is as follows: (1) the predetermined cutoff value associated with miR-486-3p is 64,800; (2) the predetermined cutoff value associated with mir-876-5p is 689; (3) the predetermined cutoff value associated with mir-381 is 30,550; (4) the predetermined cutoff value associated with miR-30c is 12,800,000; (5) the predetermined cutoff value associated with miR-432 is 19,300; and (6) the predetermined cutoff value associated with miR-15b is 1,240,000.

According to certain optional embodiments, an increase in the number of high-expression type microRNAs from the first group, or an increase in the number of the low-expression type microRNAs from the second group indicates a higher probability of a favorable prognosis.

Still optionally, an increase in the number of low-expression type microRNAs from the first group, or an increase in the number of the high-expression type microRNAs from the second group indicates a higher probability of an unfavorable prognosis.

According to some optional embodiments, the method further comprises the step of detecting the expression level (Ctₘᵢᵣ) of an additional microRNA in the non-cancerous sample. For example, the additional microRNA is selected from the group consisting of miR-15a, miR-155, miR-30b, and miR-29a.

According to other optional embodiments, the expression level (Ctₘᵢᵣ) of the microRNA is detected by real-time quantitative polymerase chain reaction (RT-qPCR) analysis.

According to yet another optional embodiment, the subject is eligible for surgical resection of hepatocellular carcinoma. Still optionally, the prognosis of the subject is a postoperative prognosis of the subject.

In some optional embodiments, the favorable prognosis is a recurrence-free survival ≧6 months, and the unfavorable prognosis is a recurrence-free survival <6 months.

In another aspect, the present disclosure is directed to a method for determining the prognosis of a subject diagnosed with hepatocellular carcinoma, based on the expression level of miR-486-3p.

According to one embodiment of the present disclosure, the method comprises the steps as follows. In step (a), a non-cancerous sample is obtained from the non-cancerous liver tissue of the subject. Then, in step (b), the expression level (Ctₘᵢᵣ) of a miR-486-3p and the expression level (Ct_{ctrl}) of miR-30d in the non-cancerous sample are detected. Next, in step (c-1) normalized expression level (ΔCtₘᵢᵣ) of the miR-486-3p is calculated according to equation (1):

normalized expression level=Ct_{ctrl}−Ctₘᵢᵣ equation (1).

Then in step (c-2), an amplified expression level (amplified ΔCtₘᵢᵣ) of the miR-486-3p is calculated according to equation (2):

amplified expression level=2^{ΔCtmir}×10⁶ equation (2).

Next, in step (c-3), the amplified ΔCtₘᵢᵣ of the miR-486-3p is compared with a cutoff value of 64,800, in which the amplified ΔCtₘᵢᵣ of the miR-486-3p being higher than the cutoff value indicates a favorable prognosis for the subject, while the amplified ΔCtₘᵢᵣ of the miR-486-3p being lower than the cutoff value indicates an unfavorable prognosis for the subject.

According to certain optional embodiments, the non-cancerous liver tissue is obtained from a paraneoplastic liver tissue of the subject.

According to other optional embodiments, the expression levels of the miR-486-3p and miR-30d are detected by real-time quantitative polymerase chain reaction (RT-qPCR) analysis.

In yet another aspect, the present invention is directed to a method for determining the prognosis of a subject diagnosed with hepatocellular carcinoma, based on the expression level of miR-432.

According to one embodiment of the present disclosure, the method comprises the steps as follows. In step (a), a non-cancerous sample is obtained from the non-cancerous liver tissue of the subject. Then, in step (b), the expression level (Ctₘᵢᵣ) of a miR-432 and the expression level (Ct_{ctrl}) of miR-30d in the non-cancerous sample are detected. Next, in step (c-1) normalized expression level (ΔCtₘᵢᵣ) of the miR-432 is calculated according to equation (1):

normalized expression level=Ct_{ctrl}−Ctₘᵢᵣ equation (1).

Then in step (c-2), an amplified expression level (amplified ΔCtₘᵢᵣ) of the miR-432 is calculated according to equation (2):

amplified expression level=2^{ΔCtmir}×10⁶ equation (2).

Next, in step (c-3), the amplified ΔCtₘᵢᵣ of the miR-432 is compared with a cutoff value of 19,300, in which the amplified ΔCtₘᵢᵣ of the miR-432 being lower than the cutoff value indicates a favorable prognosis for the subject, while the amplified ΔCtₘᵢᵣ of the miR-432 being higher than the cutoff value indicates an unfavorable prognosis for the subject.

According to certain optional embodiments, the non-cancerous liver tissue is obtained from a paraneoplastic liver tissue of the subject.

According to other optional embodiments, the expression levels of the miR-432 and miR-30d are detected by real-time quantitative polymerase chain reaction (RT-qPCR) analysis.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A and 1B provide Kaplan-Meier survival plots illustrating the relationships between the postoperative recurrence-free survival and expression levels of some microRNAs, according to one example of the present disclosure;

FIG. 2 provides Kaplan-Meier survival plots illustrating the relationships between the postoperative overall survival and expression levels of some microRNAs, according to one example of the present disclosure;

FIG. 3 provides Kaplan-Meier survival plots illustrating the combination of six microRNAs markers as a prognostic score for recurrence-free survival, according to one example of the present disclosure; and

FIGS. 4A to 4E are diagrams illustrating the expression levels of five microRNAs in non-cancerous and cancerous liver tissues in subjects with a better or poorer postoperative outcome, according to one example of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “hepatocellular carcinoma” (HCC) refers to cancer that arises from hepatocytes, as distinct from other types of hepatic cancer that may consist of liver metastases.

Here, the term “subject” or “patient” refers to an animal including the human species that is diagnosed with hepatocellular carcinoma and subject to methods of the present invention. The term “subject” or “patient” intended to refer to both the male and female gender unless one gender is specifically indicated.

The phrase “determining the prognosis” as used herein refers to the process by which the practitioner may predict the course or outcome of a condition in a subjected diagnosed with HCC. The term “prognosis” does not refer to the ability to predict the course or outcome of a condition with 100% accuracy. Instead, persons having ordinary skills in the art would understand that the term “prognosis” refers to an increased probability that a certain course or outcome will occur; that is, that a course or outcome is more likely to occur in a subject exhibiting a given condition (e.g., one or more specific microRNA expression profiles), when compared with those subjects not exhibiting the condition. A prognosis is usually made by evaluating factors or symptoms of a disease that are indicative of a favorable or unfavorable course or outcome of the disease. There are many ways that prognosis can be expressed. For example, the prognosis could be expressed in terms of overall survival (OS), recurrence-free survival (RFS), progression-free survival (PFS), and/or time to progression (TTP). OS is the amount of time from diagnosis or treatment to death, while RFS is the amount of time from complete remission to relapse of HCC or death. The durations of TTP and PFS both start from the time of treatment; but the duration of TTP only ends at the time of the progression of HCC, while the duration of PFS ends at the time of the progression of HCC or death, whichever comes first.

As used herein, the term “favorable prognosis” refers a prognosis determined for a subject having HCC which is better (i.e., has a more favorable outcome) than the prognosis for a reference subject or group of reference subjects with the same disease. For example, a patient with a favorable prognosis may be expected to exhibit a prolonged OS time or RFS time relative to reference subjects. By contrast, the term “poor prognosis” refers a prognosis determined for a subject having HCC which is worse (i.e., has a less favorable outcome) than the prognosis for a reference subject or group of reference subjects with the same disease. For example, a subject with a poor prognosis may be expected to exhibit a reduced OS time or RFS time relative to reference subjects.

The term “non-cancerous sample” shall be taken to include any sample obtained from non-cancerous liver tissues which do not contain cancer cells. However, such a “non-cancerous liver tissue” does not necessarily mean normal or healthy liver tissues, but it also includes liver tissues affected by chronic hepatitis (hepatitis B or hepatitis C) or liver cirrhosis. The “paraneoplastic liver tissue” means non-cancerous liver tissue adjacent to the HCC. For example, the paraneoplastic tissue may be taken from non-cancerous liver tissue about 1 to 5 cm away from the tumor margin.

As used herein, the term “expression level” as applied to a microRNA refers to the absolute amount or relative amount of the microRNA in the sample. According to certain embodiments of the present disclosure, the term “expression level” means the normalized level of the microRNA. Expression levels may be normalized with respect to the expression level of one or more reference (housekeeping) microRNAs (e.g., the internal control microRNA used herein), or the expression level may be normalized using global median normalization methods. Persons having ordinary skills in the art would recognize that numerous methods of normalization are known, and could be applied for use in the methods of the present disclosure. The expression level of the microRNA may be determined by any method known in the art, examples of which include but are not limited to amplification-based methods such as polymerase chain reaction (PCR), quantitative RT-PCR (qPCR), real-time quantitative PCR (RT-qPCR), semi-quantitative RT-PCR, ligase chain reaction (LCR), and strand displacement amplification (SDA).

Instead of employing conventional methods that investigate microRNAs differentially expressed between cancerous tissue and non-cancerous tissue, the present disclosure focuses on the expression profiles of microRNAs in the non-cancerous liver tissue of HCC subjects, so as to identify microRNA(s) suitable for use as a biomarker for the determination of the prognosis of such HCC subjects. Based on the identification of several microRNAs exhibiting unique expression profiles in the non-cancerous liver tissue of HCC subjects, embodiments of the present invention provides methods for determining the prognosis of a subject diagnosed with HCC. Such prognosis is useful in many aspects such as patient counsel, treatment selection, and the determination of clinical trial eligibility.

According to optional embodiments of the present disclosure, the subject is eligible for surgical resection of hepatocellular carcinoma. In this case, the prognosis of the subject could be a postoperative prognosis (e.g., OS or RFS time) of the subject.

According to embodiments of the present disclosure, the present method comprises the following steps: (a) a sampling step; (b) a detection step; and (c) a determination step. Detail discussion regarding these steps and several optional steps of the present method is provided below.

In the step (a), a non-cancerous sample is obtained from the non-cancerous liver tissue of the subject. According to optional embodiments, the non-cancerous liver tissue is obtained from a paraneoplastic liver tissue of the subject. The paraneoplastic tissue is adjacent to the tumor region, and in practice, the sample is taken from a site about 1 to 5 cm away from the tumor margin or the resected site when the tumor has been resected. In the working examples provided herein, the sampling site was about 1 cm from the resected site.

Next, in step (b), the expression level (Ctₘᵢᵣ) of at least one microRNA in the non-cancerous sample is detected. The microRNAs identified by the present inventors are categorized into two groups (i.e., a first and a second groups) based on the association of the expression profile of the microRNA and the prognosis of the subject.

According to embodiments of the present disclosure, the first group consists of three microRNAs: miR-486-3p (5′-CGGGGCAGCUCAGUACAGGAU-3′; SEQ ID No: 1), miR-876-5p (5′-UGGAUUUCUUUGUGAAUCACCA-3′; SEQ ID No: 2), and miR-381 (5′-UAUACAAGGGCAAGCUCUCUGU-3′; SEQ ID No: 3). For the first group, the expression level of any of these microRNAs is positively associated with the clinical outcome of the subject. That is, a higher expression level of a microRNA from the first group indicates a favorable prognosis, while a lower expression level of such microRNA indicates an unfavorable prognosis. Research results indicated that the expression levels of miR-15a (UAGCAGCACAUAAUGGUUUGUG; SEQ ID No: 7) and miR-29a (UAGCACCAUCUGAAAUCGGUUA; SEQ ID No: 8) are also positively associated with the clinical outcome of the subject, and hence, in certain optional embodiments, the method further comprises determining the expression level of mir-15a or mir-29a.

According to embodiments of the present disclosure, the second group consists of three other microRNAs: miR-30c (UGUAAACAUCCUACACUCUCAGC; SEQ ID No: 4), miR-432 (UCUUGGAGUAGGUCAUUGGGUGG; SEQ ID No: 5), and miR-15b (UAGCAGCACAUCAUGGUUUACA; SEQ ID No: 6). For the second group, the expression level of the microRNA is negatively associated with the clinical outcome of the subject. That is, a higher expression level of a microRNA from the second group indicates an unfavorable prognosis for the subject, while a lower expression level of such microRNA indicates a favorable prognosis. Research data revealed that the expression levels of miR-155 (UUAAUGCUAAUCGUGAUAGGGGU; SEQ ID No: 9) and miR-30b (UGUAAACAUCCUACACUCAGCU; SEQ ID No: 10) are also negatively associated with the clinical outcome of the subject, and hence, in certain optional embodiments, the method further comprises determining the expression level of miR-155 or miR-30b.

As could be appreciated, confounding factors that are non-specific to the biological conditions and non-reproducible in different experiments may be reflected in the measurement of the expression level of the microRNA. For example, even with careful control of technical variables, confounding factors that are non-specific to the HCC and non-reproducible in different experiments may result from sample-to-sample and run-to-run variations, particularly in RNA extraction efficiency and random pipetting errors. Therefore, according to certain optional embodiments of the present invention, the expression level (Ct_{ctrl}) of an internal control microRNA is also determined. The internal control microRNA may be used in a data normalization process to minimize the influence of confounding factors and improve the fidelity of the quantification process.

The internal control microRNAs are usually chosen from microRNAs with abundant and stable expression under various experimental conditions. In the working examples of the present disclosure, miR-30d is used as the internal control microRNA.

According to various embodiments of the present disclosure, the expression level of the microRNA could be determined by amplification-based methods, such as quantitative RT-PCR (qPCR), real-time quantitative PCR (RT-qPCR) and semi-quantitative RT-PCR. In particular, RT-qPCR has been widely used for quantification of gene expression that may associate with specific biomedical conditions.

Such amplification-based quantitative methods require isolation (extraction) of nucleic acids, in particular, ribonucleic acids (RNAs), from the sample. For example, phenol-based extraction is a common method for isolation of RNA. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g., precursor and mature microRNAs, 5S and 5.8S ribosomal RNA (rRNA), and U1 small nuclear RNA (snRNA)). Alternatively, extraction procedures such as those using TRIZOL™ or TRI REAGENT™, would purify all RNAs, large and small, and are efficient methods for isolating total RNA from samples that contain microRNAs and small interfering RNAs (siRNAs). Then, the extracted RNA is mixed with a suitable reaction reagents (e.g., primer, dNTP, and polymerase) mixture and subjected to amplification conditions so as to obtain amplification products. Thereafter, the amount of the amplification products is determined and used to calculate the relative expression level of the microRNA in the sample.

Afterward, in step (c), the Ctₘᵢᵣ of the microRNA or a parameter derived therefrom is compared with a cutoff value associated with the microRNA for determining the prognosis for the subject.

In the context of the present disclosure, the parameter derived from the Ctₘᵢᵣ of the microRNA may be any value calculated by an algorithm usually used by those skilled in the art. For example, the algorithm may be a normalization algorithm for reducing inter-sample variation and/or intra-sample variation. Common normalization techniques include, but are not limited to global median normalization, quantile normalization, and housekeeping normalization. According to the working examples provided herein, global median normalization and housekeeping normalization were used to normalize the Ctₘᵢᵣ data. The housekeeping normalization is employed in one optional embodiment of the present disclosure. In this optional embodiment, the expression level (Ct_{ctrl}) of the internal control microRNA described above is used as the housekeeper, and the Ctₘᵢᵣ data is normalized in accordance to the following equation (1) to obtain a normalized expression level (ΔCtₘᵢᵣ) of the microRNA:

normalized expression level(ΔCtₘᵢᵣ)=Ct_{ctrl}−Ctₘᵢᵣ equation (1).

Alternatively or additionally, the algorithm may be a function for scaling the expression level and/or determining the increase/decrease rate of the expression level. For example, a power function is used to amplify the Ctₘᵢᵣ or ΔCtₘᵢᵣ of the microRNA so that the amplified data manifest more distinction in microRNA expression profiles. According to one optional embodiment of the present disclosure, the normalized expression level (ΔCtₘᵢᵣ) of the microRNA is amplified by a power function in accordance with the following equation (2):

amplified expression level=2^{ΔCtmir}×10⁶ equation (2).

As could be appreciated, the cutoff value is microRNA-specific, which means each microRNA has a unique cutoff value associated therewith. Such cutoff value is statistically determined, as described in the working examples below. Also, it should be noted that the cutoff value is dependent on the choice of parameters (e.g., Ctₘᵢᵣ, ΔCtₘᵢᵣ, and amplified ΔCtₘᵢᵣ) for use in the comparison.

According to certain optional embodiments of the present disclosure, the internal control microRNA is miR-30d, and the amplified expression level (amplified ΔCtₘᵢᵣ) is used in the step (c) for comparison, and the cutoff values for the microRNAs suitable for use in the present method are summarized in Table 1.

TABLE 1
Cutoff values associated with amplified ΔCtₘᵢᵣ of microRNAs

| microRNA | Cutoff value for amplified ΔCtₘᵢᵣ |
|---|---|
| miR-486-3p | 64,800 |
| miR-876-5p | 689 |
| miR-381 | 30,550 |
| miR-15a | 958,600 |
| miR-29a | 1,760,000 |
| miR-30c | 12,800,000 |
| miR-432 | 19,300 |
| miR-15b | 1,240,000 |
| miR-155 | 148,600 |
| miR-30b | 3,960,000 |

Also in step (c), the microRNA is classified into a high-expression type or a low-expression type based on the results of the comparison. In particular, when the expression level Ctₘᵢᵣ of a microRNA or a parameter derived therefrom (e.g., ΔCtₘᵢᵣ, and amplified ΔCtₘᵢᵣ) is greater than the cutoff value associated with the microRNA, said microRNA is a high-expression microRNA. On the other hand, when the expression level Ctₘᵢᵣ of a microRNA or a parameter derived therefrom (e.g., ΔCtₘᵢᵣ, and amplified ΔCtₘᵢᵣ) is smaller than the cutoff value associated with the microRNA, said microRNA is a low-expression microRNA.

The determination of the prognosis for the subject is made based on the above-mentioned classification of the expression profile. Expression levels of microRNAs from the first group are positively associated with a favorable clinical outcome of the subject. Hence, the presence of a high-expression type microRNA from the first group indicates a favorable prognosis for the subject; while the presence of a low-expression type microRNA from the first group indicates an unfavorable prognosis for the subject. To the contrary, expression levels of microRNAs from the second group are negatively associated with a favorable clinical outcome of the subject. Accordingly, the presence of a low-expression type microRNA from the second group indicates a favorable prognosis for the subject; while the presence of a high-expression type microRNA from the second group indicates an unfavorable prognosis for the subject.

According to certain optional embodiments, the determination of the prognosis for the subject may also take into account other factors in addition to the expression profile of at least one microRNA. For example, according to optional embodiment, the number of microRNAs having a specified expression profile is also useful in the determination step. In particular, an increase in the number of high-expression type microRNAs from the first group, or an increase in the number of the low-expression type microRNAs from the second group indicates a higher probability of a favorable prognosis. Still optionally, an increase in the number of low-expression type microRNAs from the first group, or an increase in the number of the high-expression type microRNAs from the second group indicates a higher probability of an unfavorable prognosis.

In some optional embodiments, the favorable prognosis is a recurrence-free survival ≧6 months, and the unfavorable prognosis is a recurrence-free survival <6 months.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

The Examples below describe the analysis of microRNA expression profiles in HCC subjects and their predictive values in survival prognosis and therapeutic outcomes. For these studies, two independent cohorts of 228 total HCC patients were analyzed. The first cohort (the pilot cohort) was used in a pilot study to identify potential microRNAs associated with the survival of HCC subjects. The second cohort (the validation cohort) was used to confirm the results obtained from the pilot cohort. The data described below suggests that the expression profiles of certain microRNAs in non-cancerous tissue of HCC subjects are positively or negatively with the clinical outcomes of the subjects. Accordingly, these microRNAs are significant as biomarkers for predicting the overall survival and/or recurrence-free survival of an HCC subject.

### Materials, Methods, and Patient Characteristics

Patients and Clinical Specimens

The study involved two independent cohorts consisting of 228 HCC patients who received total removal of liver tumors from July 1998 to August 2004 in Chang Gung Medical Center, Taiwan. These patients were enrolled under the approval of the Ethics Committee of Chang Gung Memorial Hospital with written informed consent. Of the 228 patients, 12 with known better and poorer prognosis were subjected for the first-step (pilot) study. Six patients had a recurrence-free survival (RFS) time for more than 5 years (defined as a better clinical outcome) and six patients had rapid relapse within six-month after operation (defined as a clinical outcome). It is generally believed that if recurrence or metastasis is not found within five years after the initial treatment of cancer, a complete remission is achieved. Further relapse of HCC rarely occurs (a plateau is reached) if the patients have a recurrence-free survival for more than 5 years. The other 216 HCC patients including 46 females and 170 males were included for subsequent verification analysis. The basic clinical characterization of the 228 patients was listed in Table 2 and Table 3, respectively.

To avoid bias resulting from different clinical stages of the studied subjects, only subjects eligible for surgical resection (Barcelona Clinic Liver Cancer [BCLC] stage A) were enrolled. In these patients, all cancerous tissues were surgically removed, resulting in a clinically homogeneous group with no detectable malignant tumors in the liver. The non-cancerous liver tissues (paraneoplastic tissues) were taken about 1 cm from the tumor site. The cancerous liver tissues were retrieved from Institutional Tissue Bank. All samples were frozen to −70° C., immediately after surgical resection.

TABLE 2
Basic clinical characterization of 12 patients of pilot cohort

| | Prognosis | | |
|---|---|---|---|
| Clinical | Better | Poorer | |
| parameters | (n = 6) | (n = 6) | P |
| Age (years) | 49.2 ± 13.7 | 46.5 ± 8.4 | .694 |
| Sex (Male) | 4 | 5 | .999 |
| Cirrhosis | 1 | 5 | .080 |
| HBsAg positive | 5 | 6 | .999 |
| Anti-HCV positive | 1 | 0 | .999 |
| Tumor number | | | |
| 1 | 6 | 3 | .182^{a} |
| 2 | 0 | 0 | |
| 3 | 0 | 2 | |
| 4 | 0 | 1 | |
| Size | 6.6 ± 3.6 | 10.1 ± 5.0 | .199 |
| (Diameter, cm) | | | |
| Microvascular | 0 | 3 | .182 |
| invasion | | | |
| Edmondson's | | | .999 |
| grading | | | |
| 2 | 1 | 0 | |
| 3 | 4 | 6 | |
| 4 | 1 | 0 | |
| Encapsulation | 6 | 5 | .999 |
| Macrovascular | 0 | 0 | .999 |
| invasion | | | |
| Ascites | 0 | 1 | .999 |
| Alpha-fetoprotein | 850 (3-9796)^{b} | 204 (7-470) | .462^{c} |
| (ng/mL) | | | |
| Albumin (g/dL) | 4.3 ± 0.6 | 3.9 ± 0.6 | .275 |
| Bilirubin (mg/dL) | 4.1 ± 7.1 | 0.8 ± 0.3 | .282 |
| Prothrombin time | 12.0 ± 0.4 | 13.1 ± 1.0 | .031 |
| (sec) | | | |
| Creatinine | 1.8 ± 1.9 | 0.9 ± 0.2 | .275 |
| (mg/dL) | | | |
| AST (U/L) | 57.6 ± 52.1 | 83.2 ± 29.6 | .320 |
| ALT (U/L) | 59.8 ± 49.2 | 53.8 ± 33.8 | .810 |
| Alcoholism | 1 | 1 | .999 |
| Time to recur- | No recurrence | 2.8 ± 2.2 | |
| rence (months) | | | |
| Time to last | 52.8 ± 5.3 | Not assessed | |
| follow-up (months) | | | |

^{a}Comparison between patients with tumor number = 1 and those with tumor number >1.
^{b}Median (range)
^{c}Mann-Whitney test

TABLE 3
Basic clinical characterization of 216 patients of validation cohort

| | Gender | | |
|---|---|---|---|
| Clinical | Female | Male | |
| parameters | (n = 46) | (n = 170) | P |
| Age (years) | 55.6 ± 13.4 | 55.0 ± 14.8 | .804 |
| Cirrhosis | 27 (58.7%) | 90 (52.9%) | .597 |
| HBsAg positive | 31 (67.4%) | 133 (78.2%) | .183 |
| Anti-HCV positive | 19 (41.3%) | 36 (21.2%) | .010 |
| Tumor number | | | .225^{a} |
| 1 | 28 (60.9%) | 84 (49.4%) | |
| 2 | 10 (21.7%) | 37 (21.8%) | |
| 3 | 6 (13.0%) | 35 (20.6%) | |
| 4 | 2 (4.3%) | 14 (8.3%) | |
| Size (Diameter, cm) | 6.3 ± 4.9 | 6.8 ± 4.6 | .520 |
| Ascites | 4 (8.7%) | 17 (10.0%) | .791 |
| Alpha-fetoprotein | 38.5 | 44.0 | .864^{c} |
| (ng/mL) | (3.0-327500.0)^{b} | (1.5-14679.0) | |
| Albumin (g/dL) | 3.8 ± 0.6 | 3.9 ± 0.6 | .317 |
| Bilirubin (mg/dL) | 1.1 ± 1.0 | 1.5 ± 2.1 | .212 |
| Prothrombin time | 12.0 ± 1.7 | 12.4 ± 1.6 | .139 |
| (sec) | | | |
| Creatinine (mg/dL) | 1.2 ± 1.6 | 1.3 ± 1.3 | .661 |
| AST (U/L) | 91.2 ± 104.2 | 86.3 ± 126.9 | .810 |
| ALT (U/L) | 67.4 ± 65.0 | 94.2 ± 156.7 | .259 |
| Alcoholism | 3 (6.5%) | 59 (34.7%) | <.001 |

^{a}Comparison between patients with tumor number = 1 and those with tumor number >1.
^{b}Median (range)
^{c}Mann-Whitney test

RNA Isolation and RT-qPCR Analysis

Total RNAs were extracted from frozen tissues using the protocol as follows. Tissue samples (50 mg) were mixed with 1 ml of TRIZOL reagent using homogenizer. After the addition of 0.2 ml of chloroform, the homogenized sample was vortexed vigorously for 15 seconds and then incubated at room temperature (about 25-27° C.) for 2 to 3 minutes. Next, the sample was centrifuged at 12,000×g for 15 minutes at 4° C. Following the centrifugation, the upper aqueous phase of the sample was transferred into a fresh tube and mixed with 0.5 ml of isopropyl alcohol and incubated at 15 to 30° C. for 10 minutes to precipitate the RNA from the aqueous phase. After the incubation, the sample was centrifuged at 12,000×g for 10 minutes at 4° C. and whereby the RNA formed a gel-like pellet on the side and bottom of the tube. The RNA pellet was washed with 1 ml of 75% ethanol, vortexed, and centrifuged at 7,500×g for 5 minutes at 4° C. The washing procedure was repeated once and then all leftover ethanol was removed. The resultant RNA pellet was air-dried for 5-10 minutes and then dissolved in DEPC-treated water by standing on the ice for 30 minute.

The expression of mature microRNAs was measured with a stem-loop RT-qPCR method. Briefly, 10 μl RT reaction mixture containing miRNA-specific stem-loop RT primers (final concentration, 2 nM each) (SEQ ID Nos: 11-20; see, Table 4), 500 μM dNTP, 0.5 μl Superscript III (Invitrogen, Carlsbad, Calif.), 0.5 μl RNaseOut (Invitrogen), and 1 μg total RNA was used for RT reaction performed at 16° C. for 30 min, followed by 50 cycles of reaction at 20° C. for 30 s, 42° C. for 30 s, and 50° C. for 1 s. The RT products were diluted 20-fold before submitted for qPCR. Briefly, 0.5 μl of diluted RT product was used as template in a 6 μl PCR reaction mixture, which contained 1×SYBR Master Mix (Applied Biosystem, Foster City, Calif.), 200 nM miRNA-specific forward primer (SEQ ID Nos: 21-30; see, Table 4), and 200 nM universal reverse primers (CTGGTGTCGTGGAGTCGGCAATTC; SEQ ID No: 31). The condition for qPCR was 95° C. for 10 min, followed by 40 cycles of reaction at 95° C. for 15 s and 63° C. for 32 s.

TABLE 4
Stem-loop RT primers and forward primers for RT-qPCR

| microRNA | Type | Sequence (SEQ ID No) |
|---|---|---|
| miR-486-3p | RT | CTCAACTGGTGTCGTGGAGTCGGCAATTCAGTTGAGAT |
| | | CCTGTA (11) |
| | Forward | CGGCGGCGGGGCAGCTCAGTAC (21) |
| miR-876-5p | RT | CTCAACTGGTGTCGTGGAGTCGGCAATTCAGTTGAGTG |
| | | GTGATT (12) |
| | Forward | CGGCGGTGGATTTCTTTGTGAA (22) |
| miR-381 | RT | CTCAACTGGTGTCGTGGAGTCGGCAATTCAGTTGAGAC |
| | | AGAGAG (13) |
| | Forward | CGGCGGTATACAAGGGCAAGCT (23) |
| miR-30c | RT | CTCAACTGGTGTCGTGGAGTCGGCAATTCAGTTGAGGC |
| | | TGAGAG (14) |
| | Forward | CGGCGGTGTAAACATCCTACAC (24) |
| miR-432 | RT | CTCAACTGGTGTCGTGGAGTCGGCAATTCAGTTGAGCC |
| | | ACCCAA (15) |
| | Forward | CGGCGGTCTTGGAGTAGGTCAT (25) |
| miR-15b | RT | CTCAACTGGTGTCGTGGAGTCGGCAATTCAGTTGAGTG |
| | | TAAACC (16) |
| | Forward | CGGCGGTAGCAGCACATCATGG (26) |
| miR-15a | RT | CTCAACTGGTGTCGTGGAGTCGGCAATTCAGTTGAGCA |
| | | CAAACC (17) |
| | Forward | CGGCGGTAGCAGCACATAATGG (27) |
| miR-29a | RT | CTCAACTGGTGTCGTGGAGTCGGCAATTCAGTTGAGTA |
| | | ACCGAT (18) |
| | Forward | CGGCGGTAGCACCATCTGAAAT (28) |
| miR-155 | RT | CTCAACTGGTGTCGTGGAGTCGGCAATTCAGTTGAGAC |
| | | CCCTAT (19) |
| | Forward | CGGCGGTTAATGCTAATCGTGA (29) |
| miR-30b | RT | CTCAACTGGTGTCGTGGAGTCGGCAATTCAGTTGAGAG |
| | | CTGAGT (20) |
| | Forward | CGGCGGTGTAAACATCCTACAC (30) |

All qPCR reactions were performed on the ABI 7900HT Fast Real-Time PCR system (Foster City, Calif.). The ABI 7900HT SDS 2.3 software was used to calculate the threshold cycle (Ct) and relative quantification. The Ct was defined as the cycle number at which fluorescence was determined to be statistically significant above background. The sensitivity and dynamic range of the stem-loop RT-qPCR method were assessed using standard SYBR green assay for the 270 miRNA markers used in this study. Twenty-five miRNAs were excluded for further study because of low abundance (Ct>35) in the 12-sample analysis. MiR-30d (UGUAAACAUCCCCGACUGGAAG, SEQ ID No: 32) was used for tissue RNA normalization (miRNA-specific stem-loop RT primer: CTCAACTGGTGTCGTGGAGTCGGCAATTCAGTTGAGCTTCCAGT, SEQ ID No: 33; miRNA-specific forward primer: CGGCGGTGTAAACATCCCCGAC, SEQ ID No: 34).

Data Analysis and Statistical Methods

To perform an effective survey for clinically applicable prognostic miRNA marker, a total of 472 miRNAs were subjected for preliminary screening to eliminate those with extremely low levels in normal tissues. Such miRNAs with low abundance could cause difficulty in the quantitative assessment. A mixture of total RNA derived from 20 types of normal tissues was generated. To identify miRNAs which were detectable in normal tissues, the mixture of total RNAs was divided into two aliquots. In one experiment, reverse transcriptase (RTase) was removed from the reverse transcription reaction for qPCR, of which the Ct value was defined as the background (B). In the other experiment, reverse transcription was performed with RTase for qPCR, in which the Ct value was defined as a signal (S). Of the 472 miRNAs, those with ΔCt value <5 from Ct_{S}−Ct_{B}, and those with Ct value <33 were eliminated. Finally, 270 miRNAs were selected for further focused screening.

Two normalization methods were used for data analysis in the pilot study. In the first method, the raw Ct data (Ctₘᵢᵣ) were converted to 39-Ct data, which were normalized by global median normalization. The second method was housekeeping normalization in which the raw Ct data (Ctₘᵢᵣ) were normalized against the expression level (Ct_{ctrl}) of miR-30d to obtain ΔCtₘᵢᵣ in accordance with equation (1):

ΔCtₘᵢᵣ=Ct_{ctrl}−Ctₘᵢᵣ equation (1).

MiR-30d was chosen as the internal control (or housekeeper) because it exhibited low coefficient of variation (CV=0.77) and high abundance (mean Ct=24) in the liver tissues in the pilot study which profiled the expressions of 270 miRNAs. The normalized expression level (ΔCtₘᵢᵣ) was then amplified in accordance with equation (2) to obtain an amplified expression level (amplified ΔCtₘᵢᵣ):

amplified expression level=2^{ΔCtmir}×10⁶ equation (2).

In the validation stage that analyzed a validation cohort of 216 HCC patients, the housekeeping normalization method was adopted to calculate amplified expression level of the microRNAs.

In the pilot study, identification of miRNA markers with significantly differential expression levels between patients with better and poorer clinical outcomes was performed using both Mann-Whitney test and Cox proportional hazard test. Twenty miRNA markers with the lowest P values in both tests were included as candidate markers. Associations between miRNA levels and postoperative outcomes were calculated using the Kaplan-Meier method and univariate Cox regression to evaluate the differences of RFS and OS between patients who had high and low expression of candidate miRNAs. The multivariate Cox regression was executed to evaluate the joint effect of confounding factors. The experimental cutoffs were calculated according to the method of minimum P-value approach in clinical studies. For each parameter, a series of cutoffs were generated in accordance with the following equation (3):

Cutoff=Min amplified ΔCtₘᵢᵣ+n/10*(Max amplified ΔCtₘᵢᵣ−Min amplified ΔCtₘᵢᵣ), in which n=1 to 9 equation (3).

The experimental dichotomous groups were thus separated by a cutoff at least 1/10 or at most 9/10 of the factor range. This way of grouping was more readily to be used for making treatment recommendations in the future. The cutoff leading to the smallest P value was then selected for subsequent Cox proportional hazard analysis. All statistical analyses were performed with the help of SPSS version 15.0 (Chicago, Ill.).

### Example 1

### Expression Profiles of miRNA in the Non-Cancerous Liver Tissues in Patients with Known Clinical Outcomes

In the pilot stage, expression profiles of miRNA in the non-cancerous liver tissues from HCC patients with known better (n=6) and poorer (n=6) clinical outcomes were analyzed to identify potential prognostic miRNA markers. Twenty miRNA markers with the lowest P values in two statistical tests were included as candidate markers. The P values ranged from 0.010 to 0.055 using Mann-Whitney test and from 0.004 to 0.454 using Cox proportional hazard analysis.

### Example 2

### Identification of miRNA Associated with Postoperative Survivals in HCC Patients

In the verification stage, the non-cancerous liver tissues from 216 HCC patients were used to verify the prognostic predictive value of the 20 microRNA candidates. Univariate and multivariate analysis was performed to examine the association between expression levels of the 20 microRNAs and the RFS and OS, respectively, and the results were summarized in Table 5. For univariate and multivariate Cox regressions, the measures of effect are hazard ratio (HR) and adjusted hazard ratio (adjusted HR), respectively. A hazard ratio (or adjusted HR) of 1 means no effect for the high-expression group over the low-expression group. A hazard ratio (or adjusted HR) of 1.5 means that the high-expression group has 1.5 times more hazard of the low-expression group. The higher the hazard ratio (or adjusted HR) the lower is the survival probability for the high-expression group, and vice versa.

TABLE 5
Univariate and multivariate analysis of the expression levels of 20 candidate
miRNA markers for recurrence-free and overall survivals in HCC patients

| | | Recurrence-free survival | | Overall survival | |
|---|---|---|---|---|---|
| miRNA | Patient | Hazard Ratio | Adjusted HR | Hazard Ratio | Adjusted HR |
| level^{a} | No. | (95% CI) | (95% CI) | (95% CI) | (95% CI) |
| miR-151 | | | | | |
| Low | 121 | 1.004 | | 0.741 | |
| High | 95 | (0.697-1.446) | | (0.346-1.589) | |
| miR-345 | | | | | |
| Low | 159 | 0.806 | | 0.313 | |
| High | 57 | (0.525-1.239) | | (0.095-1.033) | |
| miR-374 | | | | | |
| Low | 40 | 1.302 | | 0.734 | |
| High | 176 | (0.817-2.007) | | (0.323-1.669) | |
| miR-197 | | | | | |
| Low | 122 | 0.977 | | 0.694 | |
| High | 94 | (0.678-1.407) | | (0.323-1.490) | |
| miR-29a | | | | | |
| Low | 132 | 0.839 | | 0.319 | 0.402 |
| High | 84 | (0.576-1.221) | | (0.122-0.833)^{p} | (0.146-1.105) |
| miR-155 | | | | | |
| Low | 109 | 1.577 | 2.002 | 0.823 | |
| High | 107 | (1.097-2.266)^{b} | (1.324-3.027)^{j} | (0.394-1.719) | |
| miR-15a | | | | | |
| Low | 188 | 0.459 | 0.478 | 0.387 | |
| High | 28 | (0.246-0.856)^{c} | (0.248-0.920)^{k} | (0.092-1.630) | |
| miR-381 | | | | | |
| Low | 109 | 0.653 | 0.701 | 0.543 | |
| High | 107 | (0.455-0.938)^{d} | (0.436-1.129) | (0.258-1.143) | |
| miR-432 | | | | | |
| Low | 130 | 1.625 | 1.816 | 1.279 | |
| High | 86 | (1.130-2.337)^{e} | (1.203-2.740)^{l} | (0.614-2.661) | |
| miR-486-3p | | | | | |
| Low | 104 | 0.585 | 0.543 | 0.343 | 0.502 |
| High | 112 | (0.407-0.841)^{f} | (0.330-0.893)^{m} | (0.157-0.752)^{q} | (0.218-1.154) |
| miR-30c | | | | | |
| Low | 170 | 1.713 | 1.115 | 1.258 | |
| High | 46 | (1.098-2.673)^{g} | (0.705-1.763) | (0.514-3.084) | |
| miR-101 | | | | | |
| Low | 99 | 1.223 | | 1.039 | |
| High | 117 | (0.852-1.755) | | (0.507-2.130) | |
| miR-15b | | | | | |
| Low | 86 | 1.080 | 1.074 | 1.034 | |
| High | 130 | (1.014-1.151)^{h} | (1.002-1.152)ⁿ | (0.913-1.170) | |
| miR-22 | | | | | |
| Low | 108 | 0.939 | | 0.938 | |
| High | 108 | (0.655-1.344) | | (0.458-1.925) | |
| miR-30b | | | | | |
| Low | 83 | 1.101 | 1.102 | 1.009 | |
| High | 133 | (1.031-1.175)ⁱ | (1.025-1.185)^{o} | (0.890-1.143) | |
| miR-34c-3p | | | | | |
| Low | 103 | 1.049 | | 1.300 | |
| High | 113 | (0.732-1.505) | | (0.624-2.709) | |
| miR-129-5p | | | | | |
| Low | 91 | 0.813 | | 1.110 | |
| High | 125 | (0.565-1.169) | | (0.527-2.338) | |
| miR-186 | | | | | |
| Low | 113 | 1.065 | | 0.610 | |
| High | 103 | (0.744-1.524) | | (0.290-1.285) | |
| miR-196b | | | | | |
| Low | | 84 | 0.829 | | 0.535 |
| High | 132 | (0.573-1.198) | | (0.260-1.100) | |
| miR-876-5p | | | | | |
| Low | 103 | 0.773 | | 0.474 | 0.510 |
| High | 113 | (0.539-1.107) | | (0.228-0.986)^{r} | (0.243-1.073) |

^{a}The cutoffs of high and low expression levels were determined according to the method describe in Materials and methods.
^{b}P = .014;
^{c}P = .014;
^{d}P = .021;
^{e}P = .010;
^{f}P = .004;
^{g}P = .018;
^{h}P = .017;
ⁱP = .004;
^{j}P = .001;
^{k}P = .027;
^{l}P = .015;
^{m}P = .016;
ⁿP = .043;
^{o}P = .009;
^{p}P = .020;
^{q}P = .008;
^{r}P = .046.
For other comparisons, P > .05.

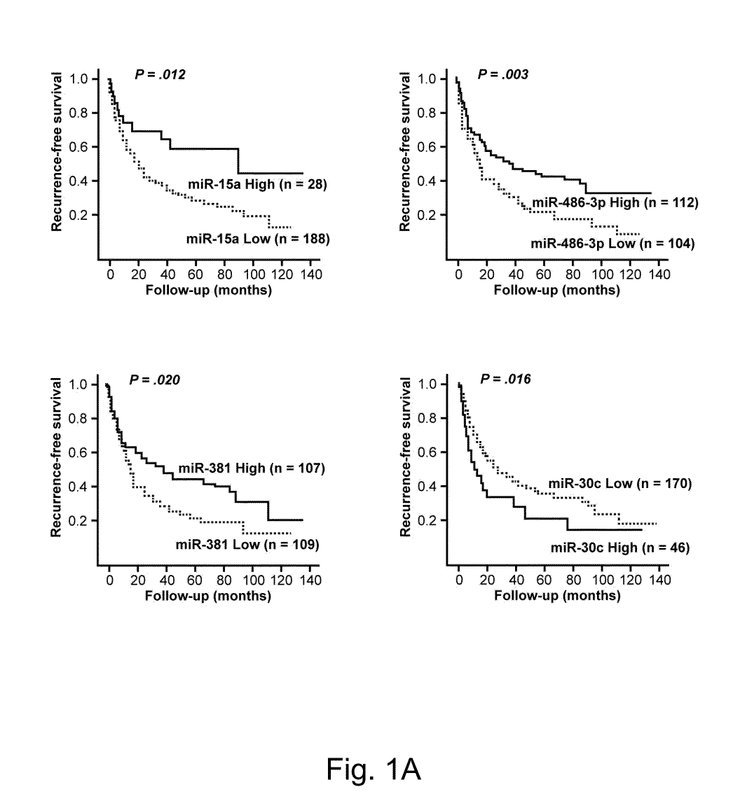

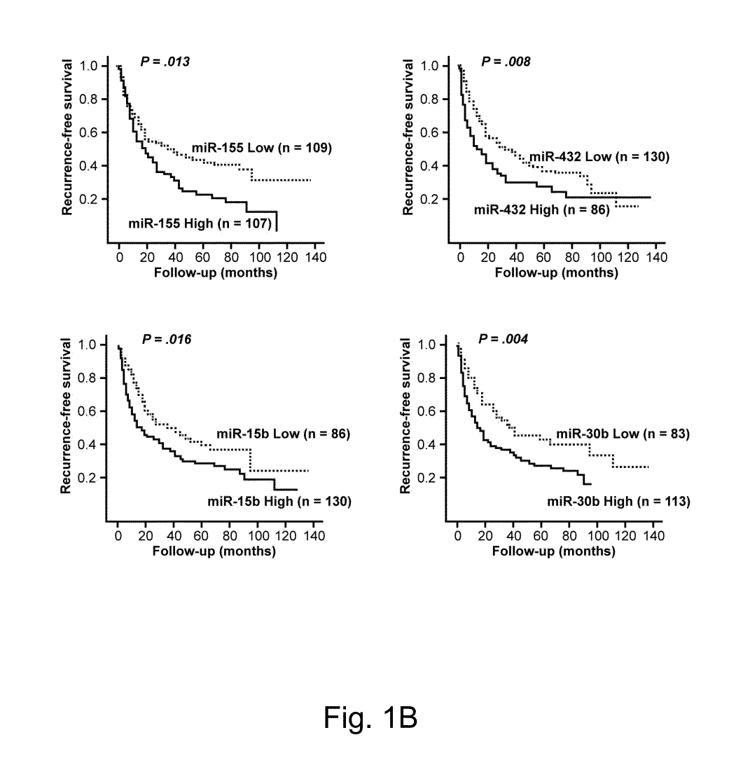

Univariate analysis, as summarized in Table 5, revealed that miR-155, miR-15a, miR-381, miR-432, miR-486-3p, miR-30c, miR-15b, and miR-30b were significantly correlated with RFS (P≦0.021 for all). The results of Kaplan-Meier survival analysis of these eight microRNAs were illustrated in FIG. 1A and FIG. 1B. As can be seen in FIG. 1A and FIG. 1B, high expression levels of miR-15a, miR-486-3p, and miR-381 are positively associated with a longer RFS with a statistical significance of P≦0.020, while high expression levels of miR-30c, miR-155, miR-432, miR-15b, and miR-30b are positively associated with a shorter RFS with a statistical significance of P≦0.016.

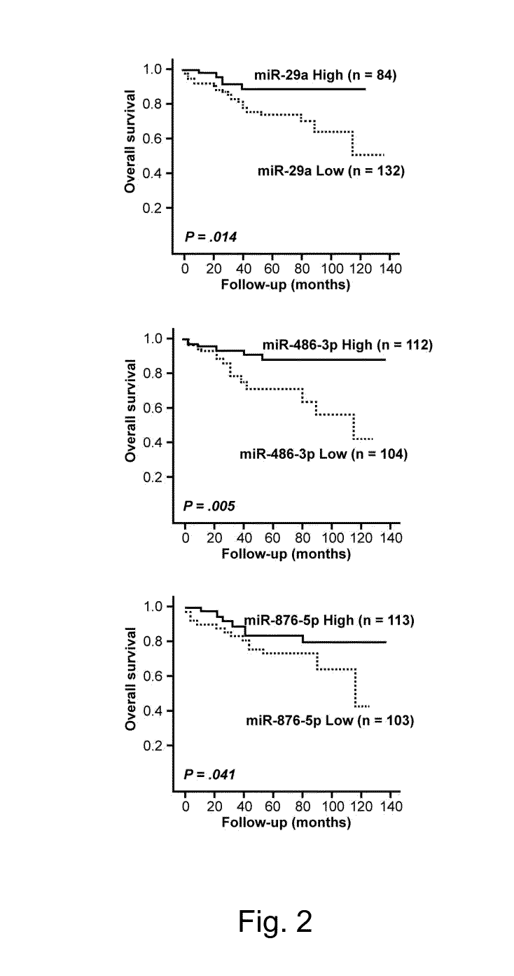

On the other hand, miR-29a, miR-486-3p and miR-876-5p were associated with OS (P≦0.046 for all). The results of Kaplan-Meier survival analysis of these three microRNAs were illustrated in FIG. 2. As can be seen in FIG. 2, high expression levels of miR-29a, miR-486-3p, and miR-876-5p were positively associated with a longer OS with a statistical significance of P≦0.041.

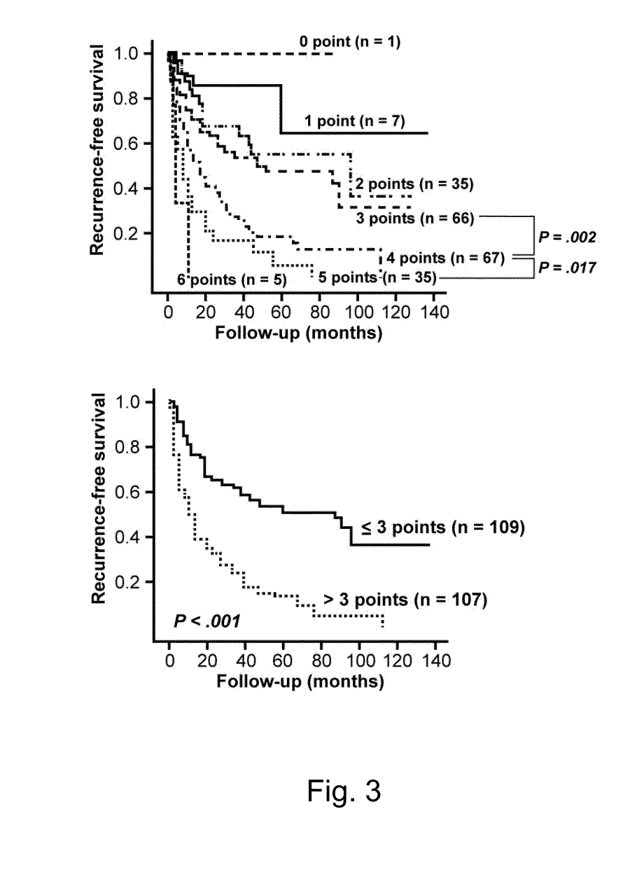

The 10 microRNAs exhibited significance in univariate Cox analysis were further subjected to multivariate Cox analysis. The results, as summarized in Table 5, indicated that 6 out of them were significantly correlated with RFS. The 6 microRNAs identified by multivariate Cox analysis were miR-15a, miR-486-3p, miR-155, miR-432, miR-15b and miR-30b. These 6 microRNAs were then combined to examine the relationship between the numbers of unfavorable microRNA factors and RFS; the results were illustrated in FIG. 3. Here, the unfavorable microRNA factor is referred to an expression profile negatively associated with RFS; for example, such unfavorable microRNA factor include low expression levels of miR-15a and miR-486-3p, as well as high expression levels of miR-155, miR-432, miR-15b and miR-30b.

Of the 216 subjects in the verification cohort, 1 (0.5%), 7 (3.2%), 35 (16.2%), 66 (30.6%), 67 (31.0%), 35 (16.2%), and 5 (2.3%) subjects had 0, 1, 2, 3, 4, 5 and 6 unfavorable miRNA factors, respectively. As illustrated in FIG. 3, the subject having zero of the 6 unfavorable miRNA factors exhibited a recurrence-free survival time of about 88 months, while the 5 subjects having all of the 6 unfavorable miRNA factors experienced recurrence in less than one year (<1, 2.3, 3.4, 4.2, and 11 months, respectively). On average, subjects having less than 3 (including 3) unfavorable miRNA factors exhibited a better recurrence-free survival than subjects having more than 4 (including 4) unfavorable miRNA factors did, and hence, subjects were stratified into two subgroups (subjects with ≦3 and >3 unfavorable microRNA factors) accordingly. Results of Kaplan-Meier analysis, as illustrated in FIG. 3, revealed that these two subgroups were statistically distinguishable regarding RFS (P<0.001).

### Example 3

### Clinicopathological Factors and microRNA Associated with Postoperative Survivals in HCC

The Cox proportional hazard model was performed to analyze whether some of the instantly claimed microRNAs (miR-155, miR-15a, miR-432, miR-486-3p, miR-15b and miR-30b) were independent factors when clinicopathological variables were included, and the results were summarized in Table 6.

TABLE 6
Univariate and Multivariate Analysis of Clinicopathological and
microRNA Parameters for Recurrence-Free Survival in HCC Patients

| | | Patient | Mean RFS | HR | Adjusted HR |
|---|---|---|---|---|---|
| | | No. | (months) | (95% CI) | (95% CI) |
| Parameter | | | | | |
| Age (years) | ≦60 | 125 | 52.9 (39.4-61.1) | | |
| | >60 | 91 | 44.8 (34.9-54.6) | 0.925 | |
| | | | | (0.638-1.341) | |
| Gender | Female | 46 | 59.3 (41.4-77.3) | | |
| | Male | 170 | 48.9 (37.1-56.2) | 1.377 | |
| | | | | (0.873-2.171) | |
| Cirrhosis | No | 99 | 56.7 (43.1-70.3) | | |
| | Yes | 117 | 41.4 (33.5-51.8) | 1.241 | |
| | | | | (0.862-1.786) | |
| Alcoholism | No | 154 | 50.9 (40.8-61.0) | | |
| | Yes | 62 | 46.9 (33.0-60.8) | 1.115 | |
| | | | | (0.758-1.640) | |
| Microvascular | No | 135 | 57.3 (46.2-68.4) | | |
| invasion | Yes | 81 | 32.5 (23.3-41.6) | 1.765 | 1.178 |
| | | | | (1.223-2.544)* | (0.787-1.765) |
| Edmondson's | I-II | 69 | 50.9 (35.6-66.2) | | |
| grading | III-IV | 147 | 48.8 (38.8-58.8) | 1.116 | |
| | | | | (0.750-1.660) | |
| Encapsulation | No | 58 | 36.4 (25.3-47.6) | | |
| | Yes | 158 | 54.0 (43.7-64.3) | 0.789 | |
| | | | | (0.533-1.168) | |
| Tumor number | 1 | 112 | 58.7 (46.6-70.7) | | |
| | >1 | 104 | 34.2 (26.3-42.3) | 1.556 | 1.596 |
| | | | | (1.082-2.238)* | (1.070-2.381)* |
| Largest tumor | ≦3 | 60 | 62.8 (48.3-77.2) | | |
| size (diameter, | >3 | 156 | 43.1 (33.5-52.7) | 1.809 | 1.431 |
| cm) | | | | (1.181-2.771)* | (0.912-2.246) |
| Macrovascular | No | 175 | 51.1 (41.6-60.6) | | |
| invasion | Yes | 41 | 45.9 (30.9-60.9) | 1.023 | |
| | | | | (0.638-1.639) | |
| Ascites | No | 195 | 52.4 (43.2-61.5) | | |
| | Yes | 21 | 25.7 (9.2-42.3) | 1.996 | 1.491 |
| | | | | (1.158-3.441)* | (0.812-2.740) |
| Serology | | | | | |
| AFP (ng/mL) | ≦25 | 96 | 57.5 (44.4-70.7) | | |
| | >25 | 120 | 43.2 (32.7-53.7) | 1.622 | 1.567 |
| | | | | (1.114-2.361)* | (1.036-2.368) |
| Albumin (g/dL) | ≦4.0 | 129 | 42.0 (31.4-52.7) | | |
| | >4.0 | 87 | 59.7 (47.0-72.4) | 0.598 | 0.723 |
| | | | | (0.411-0.868)* | (0.477-1.094) |
| Bilirubin | ≦1.2 | 152 | 54.3 (44.0-64.6) | | |
| (mg/dL) | >1.2 | 64 | 37.0 (25.6-48.3) | 1.366 | |
| | | | | (0.922-2.023) | |
| Prothrombin | ≦12 | 101 | 60.0 (46.6-73.3) | | |
| time (sec) | >12 | 115 | 41.3 (31.2-51.4) | 1.493 | 1.436 |
| | | | | (1.038-2.148)* | (0.959-2.151) |
| Creatinine | ≦1.0 | 117 | 47.0 (37.0-57.0) | | |
| (mg/dL) | >1.0 | 99 | 54.7 (40.9-68.4) | 0.917 | |
| | | | | (0.637-1.319) | |
| AST (U/L) | ≦36 | 86 | 66.1 (52.4-79.8) | | |
| | >36 | 130 | 39.8 (30.3-49.3) | 1.896 | 1.325 |
| | | | | (1.285-2.796)* | (0.837-2.095) |
| ALT (U/L) | ≦25 | 37 | 72.6 (52.0-93.3) | | |
| | >25 | 179 | 45.3 (36.4-54.1) | 2.193 | 1.287 |
| | | | | (1.207-3.982)* | (0.658-2.518) |
| Anti-HCV | Negative | 161 | 50.5 (40.4-60.6) | | |
| | Positive | 55 | 44.1 (32.6-55.6) | 0.901 | |
| | | | | (0.592-1.371) | |
| HBsAg | Negative | 52 | 47.8 (30.5-65.0) | | |
| | Positive | 164 | 50.6 (40.8-60.4) | 0.957 | |
| | | | | (0.622-1.471) | |
| miR-155 | Low | 109 | 62.3 (49.3-75.2) | | |
| | High | 107 | 35.8 (26.9-44.8) | 1.577 | 1.497 |
| | | | | (1.097-2.266)* | (0.976-2.296) |
| miR-15a | Low | 188 | 43.7 (35.5-51.9) | | |
| | High | 28 | 81.5 (56.4-106.6) | 0.459 | 0.511 |
| | | | | (0.246-0.856)* | (0.259-1.008) |
| miR-432 | Low | 130 | 53.8 (43.7-63.9) | | |
| | High | 86 | 42.1 (28.4-55.8) | 1.625 | 1.580 |
| | | | | (1.130-2.337)* | (1.005-2.484)* |
| miR-486-3p | Low | 104 | 35.9 (26.2-45.6) | | |
| | High | 112 | 63.9 (51.0-76.9) | 0.585 | 0.447 |
| | | | | (0.407-0.841)* | (0.290-0.688)* |
| miR-15b | Low | 86 | 60.3 (45.0-75.7) | | |
| | High | 130 | 42.3 (32.6-51.9) | 1.080 | 1.073 |
| | | | | (1.014-1.151)* | (0.998-1.154) |
| miR-30b | Low | 83 | 64.7 (49.8-79.7) | | |
| | High | 133 | 36.0 (28.8-43.1) | 1.101 | 1.091 |
| | | | | (1.031-1.175)* | (1.011-1.177)* |

*P < .05

Univariate analysis, as summarized in Table 6, revealed that microvascular invasion, tumor number >1, largest tumor diameter >3 cm, ascites, AFP>25 ng/mL, albumin ≦4.0 g/dL, prothrombin time >12 seconds, AST>36 U/L, ALT>25 (U/L), higher miR-155 level, lower miR-15a level, higher miR-432 level, lower miR-486-3p level, higher miR-15b level, and higher miR-30b level were significantly associated with a shorter RFS time. When these factors were included, multivariate analysis indicated that tumor number >1, higher miR-432 level, lower miR-486-3p level, and higher miR-30b level were the significant remaining factors associated with a shorter RFS time.

### Example 4

### Expression Levels of the Candidate microRNA Predictors Between Non-Tumor and Tumor Liver Tissues

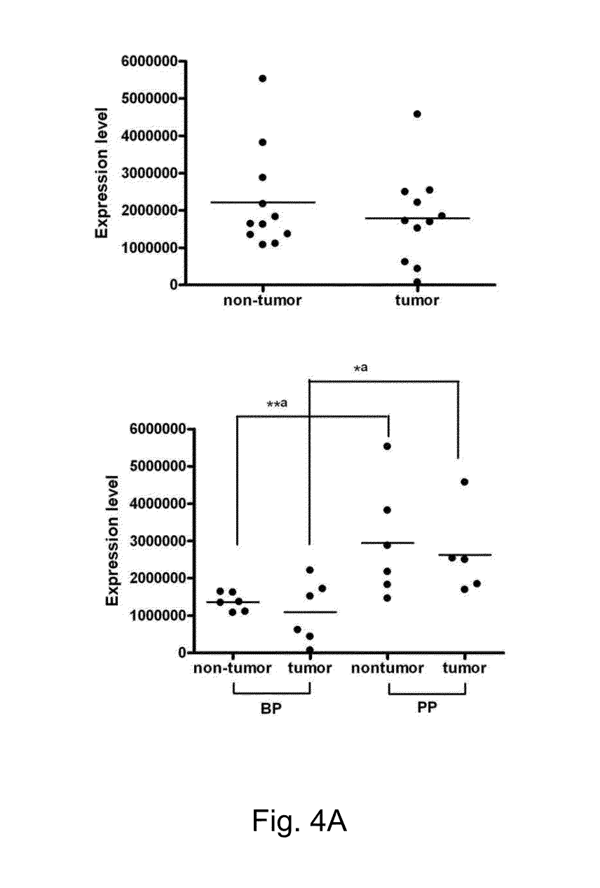

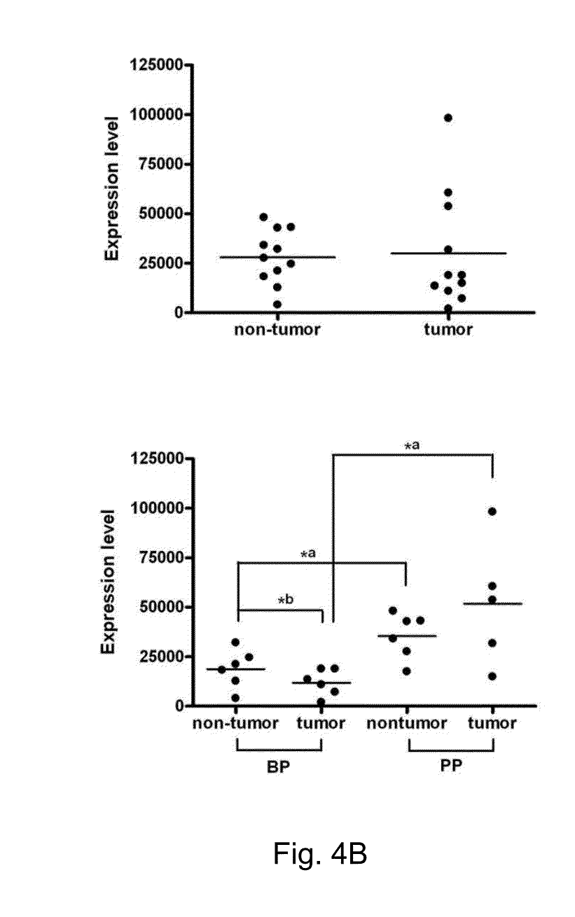

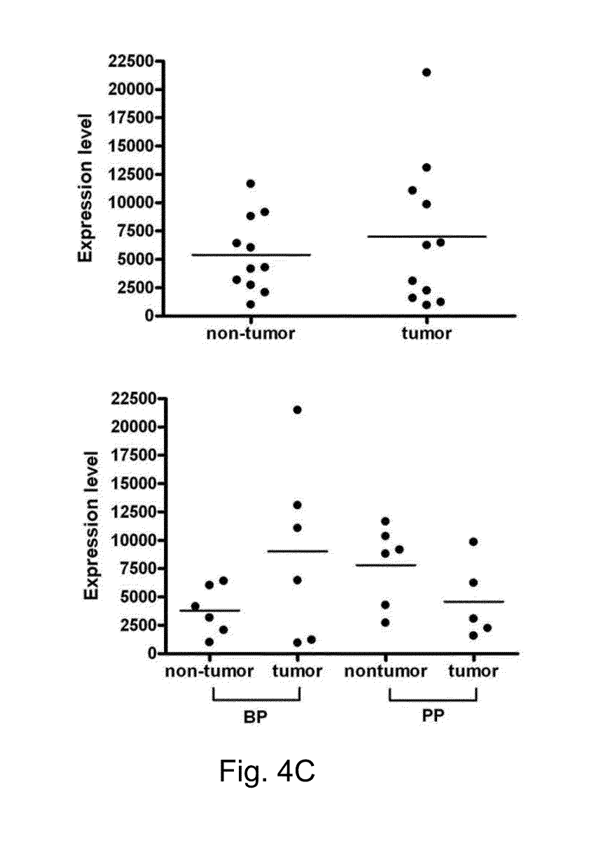

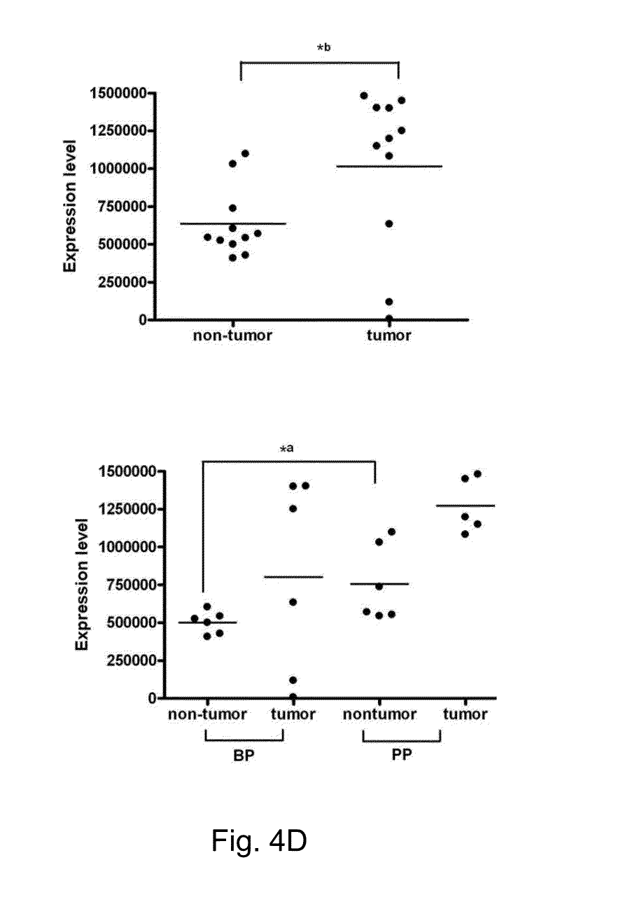

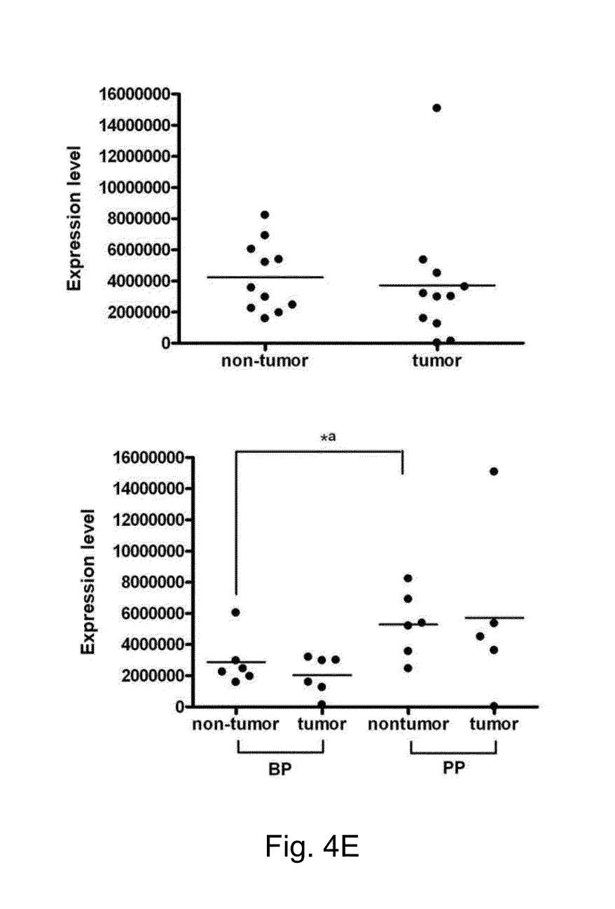

When these candidate microRNA prognostic predictors were examined, it was found that high expression levels of miR-30c, miR-155, miR-432, miR-15b, and miR-30b were associated with shorter RFS in HCC patients. To further understand the expression levels of these five microRNA predictors in liver tumor tissues of HCC patients with known postoperative outcomes, cancerous liver samples of 12 subjects in the first cohort were subjected to RT-qPCR. FIGS. 4A to 4E respectively illustrate the analyzed results of miR-30c, miR-155, miR-432, miR-15b, and miR-30b.

As could be noted in the figures, the expression levels of miR-30c (FIG. 4A, to the bottom), miR-155 (FIG. 4B, to the bottom), miR-15b (FIG. 4D, to the bottom), and miR-30b (FIG. 4E, to the bottom) in the non-cancerous liver tissues were significantly higher in subjects with poorer outcomes (PP) compared with subjects with better outcomes (BP). By contrast, for miR-432, only borderline significance was observed between PP and BP groups (P=0.055) (FIG. 4C, to the bottom).

In addition, the expression levels of miR-30c (FIG. 4A, to the bottom) and miR-155 (FIG. 4B, to the bottom) were also significantly higher in cancerous liver tissues in subjects with poorer outcomes (PP) compared with subjects with better outcomes (BP) (Mann-Whitney test, P<0.05).

For subjects with better outcomes, only the expression level of miR-155 expression was significantly decreased in the cancerous liver tissues compared with that in the non-cancerous liver tissues (Wilcoxon rank sum test, P<0.05) (FIG. 4B, to the bottom). However, if the clinical outcome was not taken into consideration, miR-155 expression levels were not significantly different between non-cancerous and cancerous liver tissues in these HCC patients (FIG. 4B, to the top).

Among the five miRNAs, only miR-15b (P<0.05) was found significantly up-regulated in the cancerous liver tissues compared to non-cancerous liver tissues (Wilcoxon rank sum test) (FIG. 4D, to the top). Therefore, if the conventional approach employing a comparison of the expression levels between cancerous and non-cancerous liver tissues were adopted (without considering the known clinical outcomes), only miR-15b could be identified by such conventional approach.

In view of the foregoing, the present disclosure provides a reliable approach for identifying microRNAs suitable for use as biomarkers for predicting the prognosis of HCC subjects. The present approach is unique at least in that it only considers the expression levels of microRNAs in the non-cancerous liver sample, thereby providing more prognostic value. By contrast, microRNA markers identified by conventional methods might have more diagnostic value because they are based on the aberrant expression of microRNAs in cancerous tissues compared with non-cancerous tissues. Since postoperative recurrent tumors usually arise from the remaining non-cancerous liver (after surgical resection), either through de novo oncogenesis or through seeding of micro-clusters of cancer cells, the cellular compositions of the non-cancerous liver thus serve as either a source of de novo cancer formation or as a suitable tissue environment for cancer cells seeding and growth. Therefore, the analysis of the molecular compositions of non-cancerous tissues provides prognostic value.

Moreover, the above data suggested that present approach is capable of identifying prognostic microRNA markers that otherwise could not be found by conventional approaches. It could be speculated that aberrant expression of some prognosis related miRNAs in the paraneoplastic tissues were associated with molecular events occurring in early stages of HCCs. These alterations might be masked by other miRNA alterations at later stages of cancer development and thus could no longer be identified by comparing cancerous and non-cancerous tissues thereafter. To date, there has been no prior publications describing or suggesting the relationship between HCC and miR-486-3p or miR-876-5p.

As discussed in the background of the invention, microRNA profiling was conducted by many groups to identify aberrantly expressed microRNAs in cancerous tissues compared with non-cancerous tissues, however, significant inconsistencies remained. In view of the data presented herein, such inconsistency still exists. For example, Gramantieri et al. (2008) disclosed that expression of mir-432 were down-regulated in HCC as compared with that of non-cancerous liver tissues; and Budhu et al. (2008) taught mir-30c and mir-15b (also known as mir-15b-2) were down-regulated in HCC as compared with that of non-tumor liver tissues. However, the results presented in Example 4 of the present disclosure suggested that the expression levels of miR-432, miR-30c or miR-15b does not differ significantly between the cancerous and non-cancerous liver tissues. Further, unlike the present disclosure, neither Gramantieri et al. nor Budhu et al. were able to identify the relationship between the unique expression profile of these microRNAs in non-cancerous liver tissues and RFS time. In addition, in a report by Chung et al. (2010), miR-15b was up-regulation in tumor tissue of HCC patients but the up-regulation was associated with a better survival, which is contradictory to our finding that a higher expression of miR-15b in non-cancerous liver tissue is positively associated with a high risk of tumor recurrence.

Toffanin et al. (2011) disclosed that miR-381 was up-regulated in subclass C3 HCC samples compared to subclasses A, B, C1 and C2 HCC samples. According to Toffanin et al., subclass C3 accounted for about 6% of all HCC cases. By contrast, the present disclosure found that the high expression level of miR-381 in non-cancerous liver tissues from HCC subjects (regardless of the subclasses thereof) is positively related to a better clinical outcome.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for determining the prognosis of a subject diagnosed with hepatocellular carcinoma, comprising,
(a) obtaining a non-cancerous sample from the non-cancerous liver tissue of the subject;
(b) detecting the expression level (Ctₘᵢᵣ) of a microRNA in the non-cancerous sample, wherein the microRNA is at least one microRNA selected from a first group consisting of miR-486-3p, miR-876-5p, and miR-381, or a second group consisting of miR-30c, miR-432, and miR-15b;
(c) comparing the Ctₘᵢᵣ of the microRNA or a parameter derived therefrom with a cutoff value associated with the microRNA for classifying the microRNA into (1) a high-expression type when the Ctₘᵢᵣ of the microRNA or the parameter derived therefrom is greater than the cutoff value associated with the microRNA, or (2) a low-expression type when the Ctₘᵢᵣ of the microRNA or the parameter derived therefrom is smaller than the cutoff value associated with the microRNA, wherein
the presence of a high-expression type microRNA from the first group indicates a favorable prognosis for the subject; while the presence of a low-expression type microRNA from the first group indicates an unfavorable prognosis for the subject; and
the presence of a low-expression type microRNA from the second group indicates a favorable prognosis for the subject; while the presence of a high-expression type microRNA from the second group indicates an unfavorable prognosis for the subject.

2. The method of claim 1, wherein the non-cancerous liver tissue is obtained from a paraneoplastic liver tissue of the subject.

3. The method of claim 1, further comprising detecting the expression level (Ct_{ctrl}) of an internal control microRNA in the non-cancerous sample.

4. The method of claim 3, wherein the internal control microRNA is miR-30d.

5. The method of claim 3, wherein the parameter derived therefrom is a normalized expression level (ΔCtₘᵢᵣ) of the microRNA calculated according to equation (1):
normalized expression level=Ct_{ctrl}−Ctₘᵢᵣ equation (1).

6. The method of claim 3, wherein the parameter derived therefrom is an amplified expression level (amplified ΔCtₘᵢᵣ) of the microRNA calculated according to equation (1) and equation (2):
normalized expression level=Ct_{ctrl}−Ctₘᵢᵣ equation (1); and
amplified expression level=2^{ΔCtmir}×10⁶ equation (2).

7. The method of claim 6, wherein
the internal control microRNA is miR-30d; and
the cutoff value associated with each of the microRNAs is as follows:
the predetermined cutoff value associated with miR-486-3p is 64,800;
the predetermined cutoff value associated with miR-876-5p is 689;
the predetermined cutoff value associated with miR-381 is 30,550;
the predetermined cutoff value associated with miR-30c is 1.28×10⁷;
the predetermined cutoff value associated with miR-432 is 19,300; and
the predetermined cutoff value associated with miR-15b is 1.24×10⁶.

8. The method of claim 1, wherein (1) an increase in the number of high-expression type microRNAs from the first group, or (2) an increase in the number of the low-expression type microRNAs from the second group indicates a higher probability of a favorable prognosis.

9. The method of claim 1, wherein (1) an increase in the number of low-expression type microRNAs from the first group, or (2) an increase in the number of the high-expression type microRNAs from the second group indicates a higher probability of an unfavorable prognosis.

10. The method of claim 1, further comprising detecting the expression level (Ctₘᵢᵣ) of an additional microRNA in the non-cancerous sample, wherein the additional microRNA is selected from the group consisting of miR-15a, miR-155, miR-30b, and miR-29a.

11. The method of claim 1, wherein the expression level (Ctₘᵢᵣ) of the microRNA is detected by real-time quantitative polymerase chain reaction (RT-qPCR) analysis.

12. The method of claim 1, wherein the subject is eligible for surgical resection of hepatocellular carcinoma.

13. The method of claim 12, wherein the prognosis of the subject is a postoperative prognosis of the subject.

14. The method of claim 1, wherein the favorable prognosis is a recurrence-free survival ≧6 months, and the unfavorable prognosis is a recurrence-free survival <6 months.

15. A method for determining the prognosis of a subject diagnosed with hepatocellular carcinoma, comprising,
(a) obtaining a non-cancerous sample from the non-cancerous liver tissue of the subject;
(b) detecting the expression level (Ctₘᵢᵣ) of miR-486-3p and the expression level (Ct_{ctrl}) of miR-30d in the non-cancerous sample;
(c-1) calculating a normalized expression level (ΔCtₘᵢᵣ) of the miR-486-3p according to equation (1):
normalized expression level=Ct_{ctrl}−Ctₘᵢᵣ equation (1);
(c-2) calculating an amplified normalized expression level (amplified ΔCtₘᵢᵣ) of the miR-486-3p according to equation (2):
amplified expression level=2^{ΔCtmir}×10⁶ equation (2); and
(c-3) comparing the amplified ΔCtₘᵢᵣ of the miR-486-3p with a cutoff value of 64,800, wherein
the amplified ΔCtₘᵢᵣ of the miR-486-3p being higher than the cutoff value indicates a favorable prognosis for the subject, and
the amplified ΔCtₘᵢᵣ of the miR-486-3p being lower than the cutoff value indicates an unfavorable prognosis for the subject.

16. The method of claim 15, wherein the non-cancerous liver tissue is obtained from a paraneoplastic liver tissue of the subject.

17. The method of claim 15, wherein the expression levels of the miR-486-3p and miR-30d are detected by real-time quantitative polymerase chain reaction (RT-qPCR) analysis.

18. A method for determining the prognosis of a subject diagnosed with hepatocellular carcinoma, comprising,
(a) obtaining a non-cancerous sample from the non-cancerous liver tissue of the subject;
(b) detecting the expression level (Ctₘᵢᵣ) of miR-432 and the expression level (Ct_{ctrl}) of miR-30d in the non-cancerous sample;
(c-1) calculating a normalized expression level (ΔCtₘᵢᵣ) of the miR-432 according to equation (1):
normalized expression level=Ct_{ctrl}−Ctₘᵢᵣ equation (1);
(c-2) calculating an amplified normalized expression level (amplified ΔCtₘᵢᵣ) of the miR-486-3p according to equation (2):
amplified expression level=2^{ΔCtmir}×10⁶ equation (2); and
(c-3) comparing the amplified ΔCtₘᵢᵣ of the miR-432 with a cutoff value of 19,300, wherein
the amplified ΔCtₘᵢᵣ of the miR-432 being lower than the cutoff value indicates a favorable prognosis for the subject, and
the amplified ΔCtₘᵢᵣ of the miR-432 being higher than the cutoff value indicates an unfavorable prognosis for the subject.

19. The method of claim 18, wherein the non-cancerous liver tissue is obtained from a paraneoplastic liver tissue of the subject.

20. The method of claim 18, wherein the expression levels of the miR-432 and miR-30d are detected by real-time quantitative polymerase chain reaction (RT-qPCR) analysis.


SEQUENCE LISTING (34 sequences)

SEQ ID NO: 1 - RNA, 21 nt, Artificial Sequence
  stem-loop RT primer
cggggcagcucaguacaggau

SEQ ID NO: 2 - RNA, 22 nt, Artificial Sequence
  stem-loop RT primer
uggauuucuuugugaaucacca

SEQ ID NO: 3 - RNA, 22 nt, Artificial Sequence
  stem-loop RT primer
uauacaagggcaagcucucugu

SEQ ID NO: 4 - RNA, 23 nt, Artificial Sequence
  stem-loop RT primer
uguaaacauccuacacucucagc

SEQ ID NO: 5 - RNA, 23 nt, Artificial Sequence
  stem-loop RT primer
ucuuggaguaggucauugggugg

SEQ ID NO: 6 - RNA, 22 nt, Artificial Sequence
  stem-loop RT primer
uagcagcacaucaugguuuaca

SEQ ID NO: 7 - RNA, 22 nt, Artificial Sequence
  stem-loop RT primer
uagcagcacauaaugguuugug

SEQ ID NO: 8 - RNA, 22 nt, Artificial Sequence
  stem-loop RT primer
uagcaccaucugaaaucgguua

SEQ ID NO: 9 - RNA, 23 nt, Artificial Sequence
  stem-loop RT primer
uuaaugcuaaucgugauaggggu

SEQ ID NO: 10 - RNA, 22 nt, Artificial Sequence
  stem-loop RT primer
uguaaacauccuacacucagcu

SEQ ID NO: 11 - DNA, 44 bp, Artificial Sequence
  stem-loop RT primer
ctcaactggtgtcgtggagtcggcaattcagttgagatcctgta

SEQ ID NO: 12 - DNA, 44 bp, Artificial Sequence
  Primer
ctcaactggtgtcgtggagtcggcaattcagttgagtggtgatt

SEQ ID NO: 13 - DNA, 44 bp, Artificial Sequence
  Primer
ctcaactggtgtcgtggagtcggcaattcagttgagacagagag

SEQ ID NO: 14 - DNA, 44 bp, Artificial Sequence
  Primer
ctcaactggtgtcgtggagtcggcaattcagttgaggctgagag

SEQ ID NO: 15 - DNA, 44 bp, Artificial Sequence
  Primer
ctcaactggtgtcgtggagtcggcaattcagttgagccacccaa

SEQ ID NO: 16 - DNA, 44 bp, Artificial Sequence
  Primer
ctcaactggtgtcgtggagtcggcaattcagttgagtgtaaacc

SEQ ID NO: 17 - DNA, 44 bp, Artificial Sequence
  Primer
ctcaactggtgtcgtggagtcggcaattcagttgagcacaaacc

SEQ ID NO: 18 - DNA, 44 bp, Artificial Sequence
  Primer
ctcaactggtgtcgtggagtcggcaattcagttgagtaaccgat

SEQ ID NO: 19 - DNA, 44 bp, Artificial Sequence
  Primer
ctcaactggtgtcgtggagtcggcaattcagttgagacccctat

SEQ ID NO: 20 - DNA, 44 bp, Artificial Sequence
  Primer
ctcaactggtgtcgtggagtcggcaattcagttgagagctgagt

SEQ ID NO: 21 - DNA, 22 bp, Artificial Sequence
  Primer
cggcggcggggcagctcagtac

SEQ ID NO: 22 - DNA, 22 bp, Artificial Sequence
  Primer
cggcggtggatttctttgtgaa

SEQ ID NO: 23 - DNA, 22 bp, Artificial Sequence
  Primer
cggcggtatacaagggcaagct

SEQ ID NO: 24 - DNA, 22 bp, Artificial Sequence
  Primer
cggcggtgtaaacatcctacac

SEQ ID NO: 25 - DNA, 22 bp, Artificial Sequence
  Primer
cggcggtcttggagtaggtcat

SEQ ID NO: 26 - DNA, 22 bp, Artificial Sequence
  Primer
cggcggtagcagcacatcatgg

SEQ ID NO: 27 - DNA, 22 bp, Artificial Sequence
  Primer
cggcggtagcagcacataatgg

SEQ ID NO: 28 - DNA, 22 bp, Artificial Sequence
  Primer
cggcggtagcaccatctgaaat

SEQ ID NO: 29 - DNA, 22 bp, Artificial Sequence
  Primer
cggcggttaatgctaatcgtga

SEQ ID NO: 30 - DNA, 22 bp, Artificial Sequence
  Primer
cggcggtgtaaacatcctacac

SEQ ID NO: 31 - DNA, 24 bp, Artificial Sequence
  Primer
ctggtgtcgtggagtcggcaattc

SEQ ID NO: 32 - RNA, 22 nt, Artificial Sequence
  Primer
uguaaacauccccgacuggaag

SEQ ID NO: 33 - DNA, 44 bp, Artificial Sequence
  Primer
ctcaactggtgtcgtggagtcggcaattcagttgagcttccagt

SEQ ID NO: 34 - DNA, 22 bp, Artificial Sequence
  Primer
cggcggtgtaaacatccccgac